(19) United States
(12) Patent Application Publication
(10) Pub. No.: US 2020/0337214 A1
(43) Pub. Date: Oct. 29, 2020

(54) A Method for Selecting and Delivering Edible Plants of a Defined Nutritional Content

(71) Applicant: Hydrogarden Ltd., Coventry (GB)

(72) Inventor: Stuart Green, Worcestershire (GB)

(21) Appl. No.: 16/960,752

(22) PCT Filed: Jan. 8, 2019
(86) PCT No.: PCT/GB2019/000005

§ 371 Date: Jul. 8, 2020

(30) Foreign Application Priority Data
Jan. 8, 2018 (GB) 1800288.1
May 18, 2018 (GB) 1808117.4
Jun. 26, 2018 (GB) 1810459.6

(51) Int. Cl. A01C 1/04; A01G 22/15; A01G 9/029; A01G 31/00; A01C 21/00

(52) U.S. Cl. CPC A01C 1/044; A01G 22/15; G06K 2209/17; A01G 31/00; A01C 21/005; A01G 9/029

(57) ABSTRACT

A method of categorising microgreens, the method comprising the steps of establishing a growing protocol for specific species of microgreen that is replicatable to deliver reliable consistent cropping and maximise vitamin and mineral availability of the species; nutritionally mapping said species through its growing cycle; calculating the nutritional content of said species for a given crop; and categorising a plurality of species of microgreen based on their nutritional load; calculating the amount of crop of the species which, on its own or in combination with a crop of a different species delivers a pre-defined vitamin or mineral load and preparing and delivering to the end user a growing media pre-seeded with the calculated amount of crop of the or each species.

### FIELD OF INVENTION

The present invention relates to a method of selecting and delivering edible plants of a defined nutritional load. More especially the invention provides a method for selecting micro-greens based on their nutritional load and delivering the micro-greens in the form of a seeded growing mat with pre-defined nutritional content.

### BACKGROUND TO THE INVENTION

Reference to micro-greens in this description is intended to mean young plants from 2 to 20 days growing period which is commonly, but not exclusively referred to as micro-greens.

Micro-greens are typically grown on a substrate that is fully controlled by the amount of seed supplied to the substrate and the environment in which they are grown, in a way that it replicable.

Typically the growing media is in the form of a mat which seed is applied and bonded to in order to form a “pre-seeded” mat for installation into growing hardware suitable to initiate germination.

Micro-greens are the richest source of nutrition available in a plant. Micro-greens grow very fast (typically around seven days) which allows crops to be turned around very rapidly making micro-greens a sustainable source of food for current and future generations.

Micro-greens are conventionally used by professional and amateur cooks, primarily as salad garnishes.

There are many published test results currently in the market concerning the nutritional content in micro-greens for specific target areas and these have been published.

Some suppliers have started offering pre-seeded mats or seed tapes for growing in the consumer's garden so that plants can be grown simply and easily in the garden of consumer. The systems being sold for home growing, and the pre-seeded mats, are aimed at the amateur and professional chef market, and cannot offer or guarantee any nutritional value.

The present applicant has been working in hydroponic horticulture for over twenty years and are a world leader in the understanding of this technology.

The applicant has accumulated considerable and extensive knowledge of growing microgreens over ten years of farming and has developed efficient growing techniques in a controlled environment under artificial light with suitable irrigation. In particular the applicant has developed and commercialised a vertical growing solution for growing plants indoors in a controlled environment utilising redundant urban and peri-urban buildings for fast and efficient full production all year round. Such a system is disclosed in, for example, the applicant's earlier patent application number WO 2015/140493.

The focus of the applicant's work, and the subject of the invention, is on the provision of selected micro-greens within the nutrition industry, as an alternative to current power and tablet vitamins and mineral supplements.

Until recently there has been little research on standardised nutritional load of micro-greens and as such there has been no reliable guaranteed growing methods available. One reason for this is that traditional horticultural practices work with too many variables.

To the best of the applicant's knowledge no one in the industry has mapped all the main varieties of microgreens across the full spectrum contents in order to ascertain a reliable and replicable yield of specific active nutrient. The main reason for the inability to collate the data is primarily due to the lack technology that provides a replicable growing cycle that guarantees the same crop growth every cycle.

Now that efficient methods of growing micro-greens have been developed by the applicant, the focus has turned to the selection of micro-greens based primarily on a calculated nutritional load and/or required nutritional benefits, and delivery of those micro-greens selected in a suitable form for rapid growth.

One aim of the invention is to develop growing protocols for around 40 key micro-greens which the applicant has identified through to trials, and to nutritionally map each micro-green to establish a ‘nutritional load’ value per crop.

The nutritional load values would be made available to nutritional experts to assist the delivery of food security and food health plans for health organisations.

The nutritional data set can then be used to create and provide pre-seeded growing mats with a defined nutritional load to match the nutritional requirements of the user. The mats would be prepared in such a way to allow them to be grown by individuals, families or communities as an alternative to vitamin supplements.

The mats can also be prepared to be grown on a commercial scale and sold, for example, as “living vitamins” into supermarkets and the like.

One aim of the invention is to commercialise the data into “food packs” consisting of a combination of micro-greens that meet the required nutritional load of the intended recipient; either as set multi vitamin type pack or to match nutritional requirements for an individual within the health care and body mapping industries.

The selection and quantity of micro-green seeds is calculated and determined using advanced computer software and algorithms processing inputted data relating the required nutritional load.

The software is pre-programed with the nutritional content of each plant. The software user is able to select the amount of vitamin or mineral dose they require and, once this date is inputted, the software processes the data to offer the requestor various options of individual crops or mix of crops to suit the requirement and indicates exact amount of the or each crop needed to grow/harvest to provide the requested dose.

A pre-seeded growing media can be prepared at a set size to match this dose requirement.

The method of apparatus would provide benefit to local councils looking to set up social enterprise and social health with systems, for example, with urban indoor allotments or with food banks producing fresh highly nutritious greens and offering employment and training. Further benefits would be provided to hospitals where a pre-defined and guaranteed nutrient load in tailored food can provide health benefits. Moreover, such methods and apparatus can be used to feed and supplement people in disaster hit areas around the globe, or in armies in war-torn areas.

The ‘nutritional load’ information and data may be used in conjunction with body mapping/nutrigenomics to allow nutritionists to tailor specific plant doses to personal diets. The outcome being predefined pre-seeded mats that would be delivered in the post to the client, for the client to grow at home to deliver them the daily dose of fresh microgreens of a target vitamin and mineral dose they require.

The general population has large numbers of people who require supplements because the vitamins and/or minerals they need are hard to get in adequate amounts in the diet.

These groups include, for example, pregnant women, nursing mothers, senior citizens, strict vegetarians or vegans, and people generally with food allergies or intolerances.

There are also many people with defined health issues and, those with diseases such as cancer, or kidney, cardiovascular, or bone disease.

Moreover, there are athletes or people training to be better at sport or who simply want to feel like they are staying healthier and who want to eat well and not consume artificial supplements.

It is anticipated that carefully selected micro-greens combinations delivered in this method and with this inventions growing technology would be able to provide guaranteed yields and nutritional content to compliment, or replace, traditional vitamin tablets.

### STATEMENT OF INVENTION

According to a first aspect of the invention there is provided a method of categorising microgreens, the method comprising the steps of:
establishing a growing protocol for specific species of microgreen that is replicatable to deliver reliable consistent cropping and maximise vitamin and mineral availability of the species;
nutritionally mapping said species through its growing cycle;
calculating the nutritional content of said species for a given crop; and
categorising a plurality of species of microgreen based on their nutritional content.

Preferably the method further comprises the step of calculating the amount of crop of the species to deliver a pre-defined vitamin or mineral content.

Preferably the method further comprises preparing and delivering to the end user a growing media pre-seeded with the calculated amount of crop of the species.

Pre-seeded mats may be commercially grown for supply to supermarkets and the like to offer pre-defined living vitamin supplements to the end consumer that have already been germinated and are growing and ready to harvest.

The end user then germinates the growing media home and harvests when ready to obtain a crop that provides their chosen amount of specified vitamins and/or minerals.

Alternatively the method further comprises preparing and delivering the calculated amount of crop of the species.

This can be provided as fresh cut crop grown from the growing media, for example a mat, or provided as a mat having the living plants grown thereon, ready for those plants to be harvested by the consumer for subsequent consumption.

According to a second aspect of the invention there is provided a method of delivering pre-seeded growing media of a defined nutritional load, comprising the following steps:
receiving a request from an end user for a defined dosage of a mineral of vitamin;
selecting one or more species of microgreen whose nutritional load value, or whose combination provides a nutritional load value, to match the requested dose level; and
calculating and outputting the size of a pre-seeded mat that would be required to provide the requested dose, or calculating and outputting the weight of the or each microgreen to harvest.

Preferably the method further comprises the step of preparing and delivering a growing media that has been pre-seeded with the selected species and calculated amount to deliver the requested dosage level.

According to a third aspect there is provided a non-transitory computer-readable storage medium having stored thereon instructions including instructions that, when executed by a processor configure the processor to identify a pre-seeded growing media of a defined nutritional load by:
receiving a request from an end user for a defined dosage of a mineral of vitamin;
selecting one or more species of microgreen whose nutritional load value, or whose combination provides a nutritional load value, to match the requested dose level; and
calculating and outputting the size of a pre-seeded mat that would be required to provide the requested dose, or calculating and outputting the weight of the or each microgreen to harvest.

According to a fourth aspect there is provided a pre-seeded growing media prepared and delivered in accordance with the methods of the first, second or third aspects.

### DETAILED DESCRIPTION OF PREFERRED EMBODIMENTS

As discussed in the background of this application, the fully controlled growing systems and apparatus developed by the applicant have allowed the applicant to provide a guaranteed replicable environment for growing microgreens.

This in turn allows the applicant to map all the main varieties of microgreens for full spectrum contents to then formulate a strategy to raise selected key actives that are found in high amounts.

The data can then be used to calculate and select a specific growing area of mat that will yield a certain amount of target active such as specific vitamins and minerals.

If a selected crop of microgreens, chosen for its particular nutrient, is sown onto the mat at the same ratio every time and grown in a guaranteed replicable environment, then a guaranteed nutritional load can be achieved.

For example, if red cabbage microgreen is found to give 100 mg of vitamin C per 100 g of harvested leaf then the data can be used to calculate the size of the mat needed to grow that 100 g and also how many seeds need to be bonded to the mat.

For this example, if 100 g of crop is required, then the number of seeds and the surface area of the mat can be calculated. For example, the area required may be 10 cm², then a consumer requiring 200 mg of vitamin C can be provided with a 20 cm² on a pre-seeded cabbage mat and germinate the mat within a controlled growing chamber that is pre-set to the calculated growing protocol for that cabbage. The temperature, lighting and overall environment within the growing chamber is set to grow red cabbage providing the required nutritional load.

Changing the environment or lighting of the chamber will provide a different result on the nutrition content and yield.

The general process behind the formulation of nutritional load data for various species of micro-greens has been described above.

Implementation of systems and apparatus that utilises the nutritional load data will now be described.

Once the nutritional data on the micro-greens is established, trials for each key crop are undertaken to see how, through manipulation of the environment in which they grow, the nutritional load can be increased or enhanced. Trials will involve, for example without limitation, experimenting with different environmental controls, positive and negative pressures, light amendments and feed regimes.

Once the nutritional levels have been mapped, all values will be put into a software algorithm. This algorithm selects options of microgreens that can deliver a specified dose of target element (vitamin/mineral). The software will specify what elements are in each selection. The software will process the data to mix and match for the best selection of either single variety or mixed varieties to deliver the target dose of vitamins and minerals. The end-user s can then select what is the best array of vitamins and minerals for their needs from the shortlist provided by the algorithm.

For health professionals, the software will include the ability to deselect selections high in unwanted elements that are important for patient care.

A cancer specialist may for example, provide a diet plan for a patient who requires high levels of vitamin B12 and magnesium with high anti-oxidant properties but low copper, by choosing the best ratio of nutrients from a number of different options of combinations. The specialist may mix different crops for example to combine a crop with a high vitamin B12 with another crop having high anti-oxidant levels. Following this, the specialist can also set up mixes for multi vitamin sets which could be for example 10 cm² of one crop from the pre-seeded mat and 20 cm² of another crop from the pre-seeded mat to provide the optimum nutrient ratio to meet the needs of the patient.

Crops may be grown to compliment others acting as a ‘catalyst’ crop to the target crop making it more effective when consumed.

The mats may be pre-fertilised or pre-inoculated with beneficial bacteria depending on the method for increase the nutritional load.

Experiments and trials will take place utilising vertical growing solutions, such as that developed by the applicant and previously referred to, within a 20 ft containerised room. The containerised room takes the form of foldable and modular structure. The containerised room is constructed in this form so that it is adaptable to the variable environmental controls and area required. The modular structure of the room also allows two units to be transported in a 40 ft container and flown via helicopter to places that are difficult to reach where the containers can then be erected and fully operational to grow crops within one day.

The modular structure of the containers allow them to be easily transported and quickly erected, in for example a village. The systems, when operational provide a defined and guaranteed nutritional value range and yield, which may have been previously specified for their purpose.

Other smaller modular room systems would be available for delivery and erection in places such as solutions of urban allotment projects, food banks, hospitals, prisons, schools and other social enterprise projects.

Finally, simple “in-home” systems would be available to provide a low-cost home-growing solution in the form of a tray with pre-seeded mats of specific nutritionally loaded greens ready for juicing in the kitchen.

For small scale commercial users and home use, the invention provides for the preparation of pre-seeded mats of specific nutritional content that will either be sold as a mat that the end user germinates and grows in one of the specified systems that can guarantee consistency, or it will be sold as ready grown crop on the mat that customers can buy from retailers just like any other vegetable crop.

Pre-seeded growing media or living growing crops will also be sold at set dose levels so the packaging defines the dose level then the customer can choose how much to take. For example, harvesting a 10 cm strip of set growing media will yield 10 mg of vitamin C. Harvesting 20 cm will yield 20 mg of vitamin C.

Then once ready to harvest the end user will harvest a specific amount of the crop that is detailed on the packaging to deliver the required dose. For example, the end user wants 10 mg of vitamin C so they harvest 20 cm of the growing mat for that amount.

Specialist growing fertilisers can be provided to assist in the high level of nutritional load to be achieved.

It will be appreciated that the foregoing is merely an example of an embodiment and just some examples of its use. The skilled reader will readily understand that modifications can be made thereto without departing from the true scope of the invention.

1. A method of categorizing microgreens, the method comprising the steps of:
establishing a growing protocol for specific species of microgreen that is replicable to deliver reliable consistent cropping and maximize vitamin and mineral availability of the species;
nutritionally mapping said species through its growing cycle;
calculating the nutritional content of said species for a given crop; and
categorizing a plurality of species of microgreen based on their nutritional load.

2. A method according to claim 1, further comprising the step of calculating the amount of crop of the species which, on its own or in combination with a crop of a different species delivers a pre-defined vitamin or mineral load.

3. A method according to claim 2, further comprising the step of preparing and delivering to the end user a growing media pre-seeded with the calculated amount of crop of the or each species.

4. A method according to claim 2, further comprising preparing and delivering the calculated amount of crop of the species.

5. A method of delivering pre-seeded growing media of a defined nutritional load, comprising the following steps:
receiving a request from an end user for a defined dosage of a mineral of vitamin;
selecting one or more species of microgreen whose nutritional load value, or whose combination
provides a nutritional load value, to match the requested dose level; and
calculating and outputting the size of a pre-seeded mat that would be required to provide the requested dose, or calculating and outputting the weight of each microgreen to harvest.

6. A method according to claim 5, further comprising the step of preparing and delivering a growing media that has been pre-seeded with the selected species and calculated amount to deliver the requested dosage level.

7-8. (canceled)

9. A non-transitory computer-readable storage medium having stored thereon instructions including instructions that, when executed by a processor configure the processor to identify a pre-seeded growing media of a defined nutritional load by:
receiving a request from an end user for a defined dosage of a mineral of vitamin;
selecting one or more species of microgreen whose nutritional load value, or whose combination provides a nutritional load value, to match the requested dose level; and
calculating and outputting the size of a pre-seeded mat that would be required to provide the requested dose, or calculating and outputting the weight of each microgreen to harvest.
